(19) United States
(12) Patent Application Publication
(10) Pub. No.: US 2018/0042324 A1
(43) Pub. Date: Feb. 15, 2018

(54) CAP WITH INTEGRATED AND INTERCHANGEABLE EYE PROTECTION

(71) Applicant: Bradley W. King, Elmvale (CA)

(72) Inventor: Bradley W. King, Elmvale (CA)

(21) Appl. No.: 15/233,931

(22) Filed: Aug. 10, 2016

(51) Int. Cl. A42B 1/24; A42B 1/06; A61F 9/04

(52) U.S. Cl. CPC A42B 1/247; A61F 9/04; A42B 1/062

(57) ABSTRACT

A cap eye protection device comprising: a) a cap member comprising a brim with a plurality of brim fasteners capable of engaging with a plurality of fasteners of an eye protection lens on an underside of the brim; and b) a transparent eye protection lens comprising a plurality of lens fasteners capable of engaging with the plurality of brim fasteners, and a plurality of connector tabs formed on a top side of the eye protection lens, wherein the plurality of connector tabs are each engaged with the plurality of lens fasteners, wherein the eye protection lens is comprised of a flexible plastic and is molded to conform to a concave shape of an underside of the brim, wherein the eye protection lens is interchangeable, and wherein the brim fasteners and lens fasteners allow for interchanging eye protection lenses between a clear lens and a tinted lens.

### FIELD OF THE INVENTION

The present disclosure is in the field of caps and hats with integrated eye protection and interchangeable eye protection systems.

### BACKGROUND OF THE DISCLOSURE

During sporting, recreational and outdoor activities many individuals feel it is necessary to wear a hat or cap to protect the scalp, nose and eyes. However, during these activities sunglass and/or eye protection is used in conjunction with a hat or cap. As such, it is desirable and convenient to employ sunglass and/or eye protection affixed to a brim of a hat or cap that extends longitudinally forward from the hat or cap portion. Examples include the attachment of eyewear devices to a visor, rim or brim portion of baseball caps, hard hats, and various other types of caps or hats. However, currently available visor or brim attached eyewear devices are often plagued by functional and structural obstacles.

Therefore, there is a need for a resilient and functional cap affixed with interchangeable eye protection.

### SUMMARY OF THE INVENTION

Disclosed herein is a cap eye protection device comprising: a) a cap member comprising a brim with a plurality of brim fasteners capable of engaging with a plurality of fasteners of an eye protection lens on an underside of the brim; and b) a transparent eye protection lens comprising a plurality of lens fasteners capable of engaging with the plurality of brim fasteners, and a plurality of connector tabs formed on a top side of the eye protection lens, wherein the plurality of connector tabs are each engaged with the plurality of lens fasteners, wherein the eye protection lens is comprised of a flexible plastic and is molded to conform to a concave shape of an underside of the brim, wherein the eye protection lens is interchangeable, and wherein the brim fasteners and lens fasteners allow for interchanging eye protection lenses between a clear lens and a tinted lens.

In another aspect, disclosed herein is a kit comprising a cap eye protection device comprising: a) a cap member comprising a brim with a plurality of brim fasteners capable of engaging with a plurality of fasteners of an eye protection lens on an underside of the brim; and b) a transparent eye protection lens comprising a plurality of lens fasteners capable of engaging with the plurality of brim fasteners, and a plurality of connector tabs formed on a top side of the eye protection lens, wherein the plurality of connector tabs are each engaged with the plurality of lens fasteners, wherein the eye protection lens is comprised of a flexible plastic and is molded to conform to a concave shape of an underside of the brim, wherein the eye protection lens is interchangeable, and wherein the brim fasteners and lens fasteners allow for interchanging eye protection lenses between a clear lens and a tinted lens.

### BRIEF DESCRIPTION OF THE DRAWINGS

FIG. 1 is an exploded view of an interchangeable cap eye protection device 100 comprising a transparent eye protection lens 110.

FIG. 2 is a perspective view of an interchangeable cap eye protection device 200 comprising a plurality of lens fasteners 112, 114 and 116, whereby the eye protection lens 110 has a top edge 126 is molded to conform to a concave shape 204 of an underside 124 of the brim 104.

FIG. 3A is a perspective view of an eye protection lens 300 comprising connector tabs 118, 120 and 122.

FIG. 3B is a perspective view of an eye protection lens 400 comprising a custom design tint 408.

FIG. 4A is a perspective view of an interchangeable cap eye protection device 500 illustrating a topside 516 of the brim 104 comprising brim fasteners comprising brim cap snaps 510, 512 and 514 which are recessed via recesses 518, 520 and 522.

FIG. 4B is a perspective view of an interchangeable cap eye protection device 600 illustrating an underside 124 of the brim 104 comprising brim snaps 128, 130 and 132 and apertures 502, 504 and 506 and showing a brim cap snap 510 engaged 508 with brim snap 132 via aperture 506.

### DETAILED DESCRIPTION OF THE EMBODIMENTS

The following is a detailed description of certain specific embodiments of the cap eye protection devices and the cap eye protection device kits disclosed herein.

In one aspect, disclosed herein is a cap eye protection device comprising: a) a cap member comprising a brim with a plurality of brim fasteners capable of engaging with a plurality of fasteners of an eye protection lens on an underside of the brim; and b) a transparent eye protection lens comprising a plurality of lens fasteners capable of engaging with the plurality of brim fasteners, and a plurality of connector tabs formed on a top side of the eye protection lens, wherein the plurality of connector tabs are each engaged with the plurality of lens fasteners, wherein the eye protection lens is comprised of a flexible plastic and is molded to conform to a concave shape of an underside of the brim, wherein the eye protection lens is interchangeable, wherein the brim fasteners and lens fasteners allow for interchanging eye protection lenses between a clear lens and a tinted lens, and wherein the cap is a baseball cap.

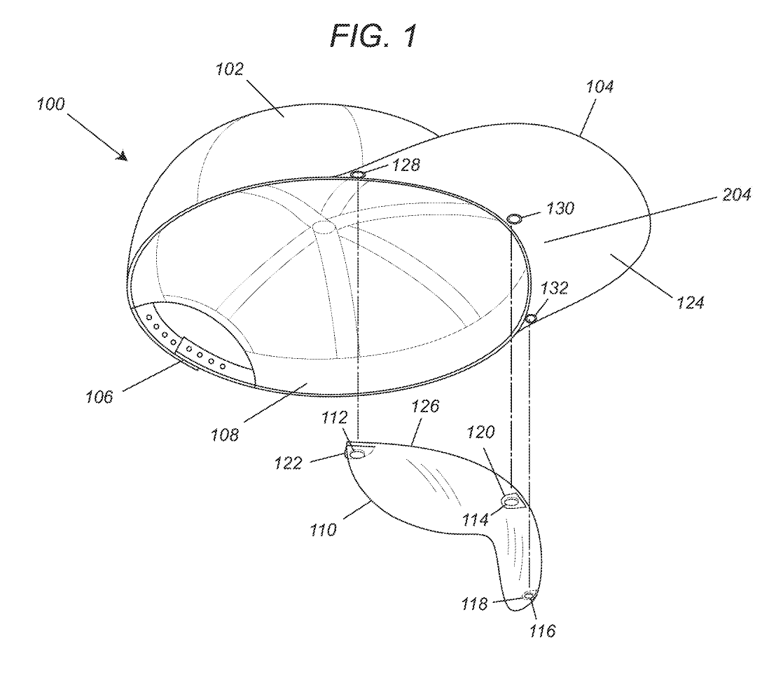

Turning to the drawings, FIG. 1 depicts an exploded view of a cap eye protection device 100 as disclosed herein. In some embodiments, the cap portion 102 comprises a fitted size, or alternatively may have a hook and loop size adjustment, strap and buckle size adjustment or a tab and snap size adjustment 106. The exploded view of the interchangeable cap eye protection device 100 comprising a transparent eye protection lens 110 that has a top edge 126 that is molded to fit against a concave shape 204 of an underside 124 of the brim 104. In some embodiments, the eye protection lens is tinted. In some embodiments, the eye protection lens is selected from clear, mirrored tinted, yellow tint, red tint, orange tint, blue tint, light smoke, dark smoke and/or a custom design tint. In some embodiments, the eye protection lens is treated for ultraviolet protection. Moreover, in some embodiments the eye protection lens 110 comprises connector tabs 118, 120 and 122 which are positioned at about a 90 degree angle and angled towards the cap portion 102 relative to the eye protection lens 110. As illustrated, the eye protection lens 110 is affixed to the brim 104 adjacent to the cap head band 108 and distributed across the top edge 126 of the eye protection lens 110. As shown with FIG. 1, brim fasteners 128, 130 and 132 are affixed to the brim 104, whereby lens fasteners 112, 114 and 116 are capable of engaging and disengaging with brim fasteners 128, 130 and 132. In this manner, brim fasteners 128, 130 and 132 are affixed to the brim 104 and lens fasteners 112, 114 and 116 are affixed to the connector tabs 118, 120 and 122 of the eye protection lens 110.

The eye protection lens 110 is made from sheet of uniform thickness with curved lower edges at each side of the nose-cutout portion 202. The eye protection lens 110 may also have a nose-bridge portion fitted to the lower edge of the nose-cutout portion, and preferably constructed from plastic, rubber and/or foam rubber. The eye protection lens 110 may be derived from a sheet of polycarbonate material, whereby the eye protection lens 110 derived from a sheet of polycarbonate material has a thickness between about 0.25 mm and 1.5 mm. Moreover, the eye protection lens 110 may be molded with at suitable mold and a polycarbonate material or other suitable lens material, whereby the molded eye protection lens 110 has a thickness between about 1.2 mm and 1.5 mm. In some embodiments, the cap eye protection device further comprises that the eye protection lens is comprised of a flexible plastic and the surface is molded to conform to about a curvature of a rim of a cap.

The cap may be a hat or cap or partial cap. The brim 104 may be of continuous annular form in the case of a full hat, but may be at the front only as in the case of a cap as disclosed herein. The crown of the hat or cap may be partially open or a baseball-type cap body, having the front brim 104 of stiffened and flexible material. The hat, cap or partial cap, with the head band 108 being discontinued at the rear to be replaced by adjustable connecting straps.

As disclosed herein the eye protection lens 110 is secured to the brim 104 via the lens fasteners 112, 114 and 116 and brim fasteners 128, 130 and 132 and that upon removal of the cap or hat from the user's head, the eye protection lens 110 is removed with the brim 104 of the cap or hat. Moreover, the eye protection lens 110 may be removed and/or replaced, whereby the lens fasteners 112, 114 and 116 are capable of being disengaged and reengaged with the brim fasteners 128, 130 and 132.

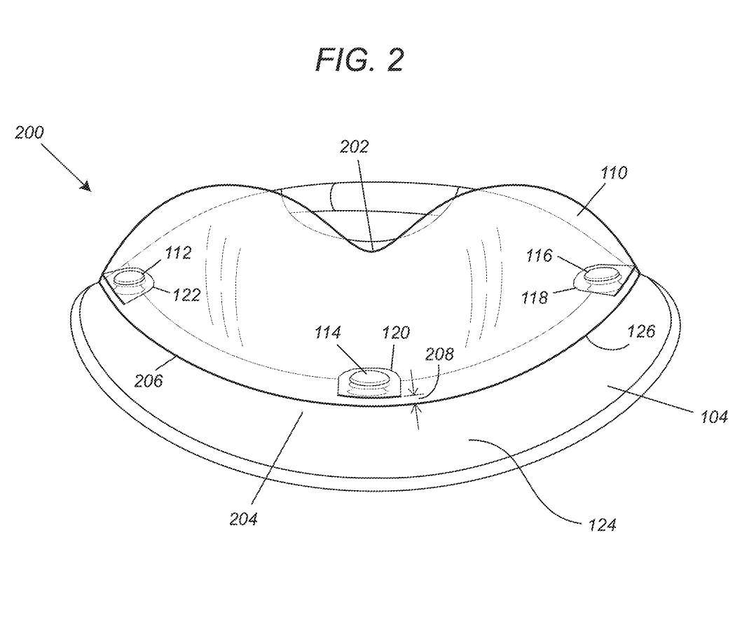

Turning to FIG. 2, illustrates a perspective view of an interchangeable cap eye protection device 200 comprising a plurality of lens fasteners 112, 114 and 116, whereby the eye protection lens 110 has a top edge 126 is molded to conform to a concave shape 204 of an underside 124 of the brim 104. As shown, the eye protection lens 110 is shaped to have a profile accommodating a user's nose. Moreover, the transparent eye protection lens 110 that has a top edge 126 that is molded to fit against a concave shape 204 of an underside 124 of the brim 104. In some embodiments, the cap eye protection device further comprises that the eye protection lens is comprised of a flexible plastic and the top edge is molded to conform to a concave shape of an underside of the brim to reduce or eliminate debris, wind and/or sunlight from penetrating a seam 206 between the eye protection lens and the underside of the brim. FIG. 2 depicts that the connector tabs 118, 120 and 122 may be affixed at a distance 208 from the top edge 126 between about 0.1 mm and 5 mm, whereby the connector tabs 118, 120 and 122 are affixed or sandwiched by the lens fasteners 112, 114 and 116 to allow for ease attachment and detachment of the eye protection lens 110. In some embodiments, the connector tabs 118, 120 and 122 may be affixed at a distance 208 from the top edge 126 of about 3 mm. In some embodiments, the connector tabs 118, 120 and 122 are aligned at an angle 410 between about 60 degrees to 120 degrees relative to the eye protection lens surface 310. In some embodiments, the brim and eye protection lens connect via three pairs of fasteners and three connector tabs.

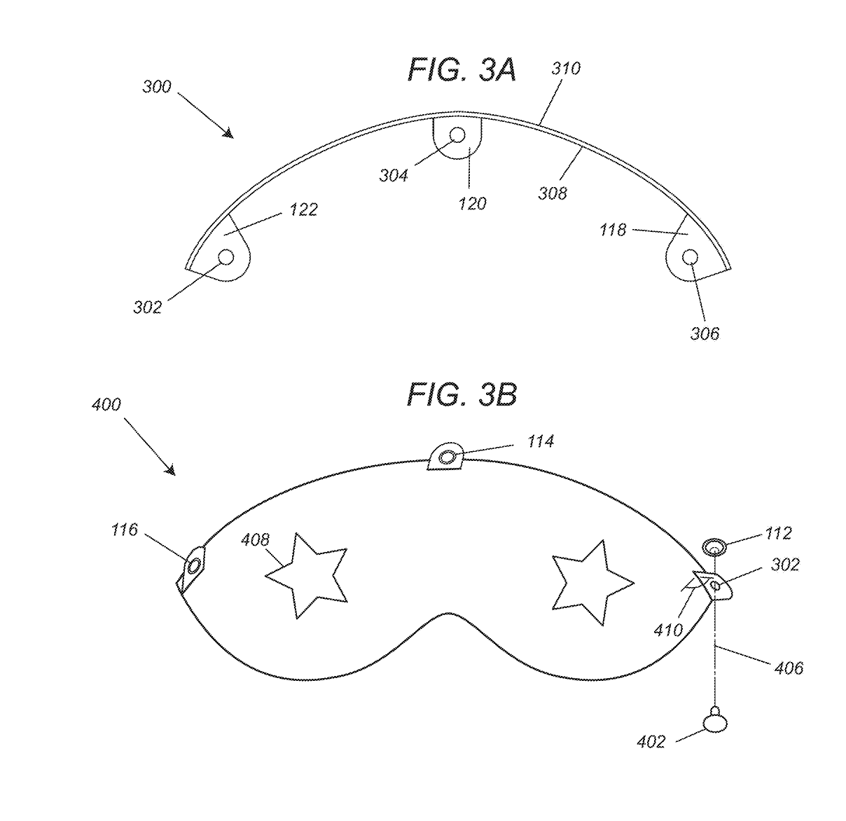

Turning to FIG. 3A, the eye protection lens 300 comprises connector tabs 118, 120 and 122. Moreover, the eye protection lens 300 comprises a surface 310 that is molded to conform to about a curvature 308 of a rim of a cap. In this manner, the eye protection lens 300 and the connector tabs 118, 120 and 122 are positioned to be hidden beneath the underside 124 of the brim 104 as shown with FIGS. 1 and 2. FIG. 3B is a perspective view of an eye protection lens 400 comprising a custom design tint 408. As shown, the lens fasteners 112, 114 and 116 may be comprised of a lens dome snap 402 and a lens snap 112 which are engaged 406 with the eye protection lens 400 via apertures 302. In this manner, the lens dome snap 402 and the lens snap 112 are snapped together and affixed to and sandwiching each of the connector tabs 118, 120 and 122, respectively. In some embodiments, the connector tabs each comprise a mounting hole capable of engaging with a fastener.

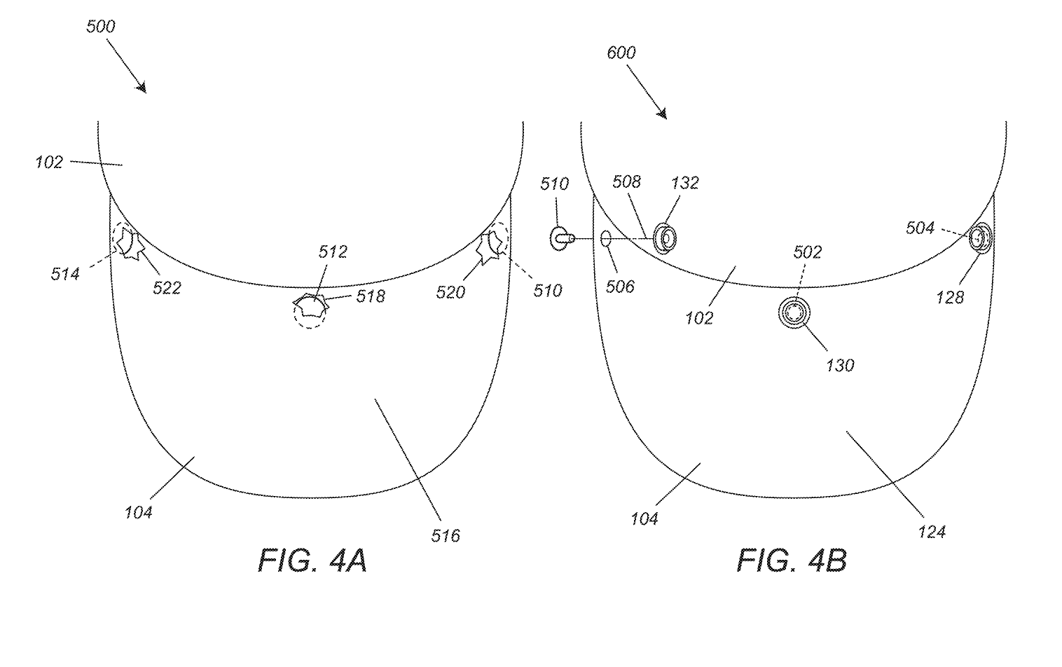

FIG. 4A is a perspective view of an illustration of a topside 516 of a brim 104 comprising brim fasteners comprising brim cap snaps 510, 512 and 514 which are recessed within the brim 104 via recesses 518, 520 and 522, whereby the brim cap snaps 510, 512 and 514 are not visible from the topside 516 of a brim 104. In some embodiments, the brim cap snaps 510, 512 and 514 are recessed via recesses 518, 520 and 522 of the brim 104.

FIG. 4B is a perspective view of an illustration of an underside 124 of the brim 104 comprising brim snaps 128, 130 and 132 and apertures 502, 504 and 506 and showing a brim cap snap 510 engaged 508 with brim snap 132 via aperture 506. The brim snaps 128, 130 and 132 may be engaged 508 with the brim cap snaps 510, 512 and 514 of FIGS. 4A and 4B, via apertures 502, 504 and 506. In some embodiments, the brim fasteners and the corresponding lens fasteners are metal snaps, wherein the lens fasteners are capable of being engaged and disengaged with the brim fasteners.

In another aspect, disclosed herein is a kit comprising a cap eye protection device comprising: a) a cap member comprising a brim with a plurality of brim fasteners capable of engaging with a plurality of fasteners of an eye protection lens on an underside of the brim; and b) a transparent eye protection lens comprising a plurality of lens fasteners capable of engaging with the plurality of brim fasteners, and a plurality of connector tabs formed on a top side of the eye protection lens, wherein the plurality of connector tabs are each engaged with the plurality of lens fasteners, wherein the eye protection lens is comprised of a flexible plastic and is molded to conform to a concave shape of an underside of the brim, wherein the eye protection lens is interchangeable, wherein the brim fasteners and lens fasteners allow for interchanging eye protection lenses between a clear lens and a tinted lens, and wherein the cap is a baseball cap.

In the manner, the kit as disclosed herein may comprise a variety of eye protection lens (i.e., clear, tinted, etc.) with lens fasteners affixed to each lens, whereby the user may conveniently remove and replace lenses, as needed. In some embodiments, the kit further comprises an additional plurality of brim fasteners and lens fasteners. In some embodiments, the eye protection lens is a plurality of lenses selected from clear, mirror tinted, yellow tint, red tint, orange tint, blue tint, light smoke, dark smoke and/or a custom design tint. In some embodiments, the eye protection lens is treated for ultraviolet protection. In some embodiments, the brim and eye protection lens connect via three pairs of fasteners and three connector tabs. In some embodiments, the connector tabs each comprise a mounting hole capable of engaging with a fastener. In some embodiments, the connector tabs are aligned at about a 90 degree angle relative to the eye protection lens. In some embodiments, the brim fasteners and the corresponding lens fasteners are metal snaps, wherein the lens fasteners are capable of being engaged and disengaged with the brim fasteners.

### Definitions

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained. It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. As used herein, the term “comprising” means including elements or steps that are identified following that term, but any such elements or steps are not exhaustive, and an embodiment can include other elements or steps.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Many modifications and variations can be made without departing from its spirit and scope, as will be apparent to those skilled in the art. Functionally equivalent methods and devices within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods or devices, which can of course vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict any definitions in this disclosure.

What is claimed is:

1. A cap eye protection device comprising:
a) a cap member comprising a brim with a plurality of brim fasteners capable of engaging with a plurality of fasteners of an eye protection lens on an underside of the brim; and
b) a transparent eye protection lens comprising a plurality of lens fasteners capable of engaging with the plurality of brim fasteners, and a plurality of connector tabs formed on a top side of the eye protection lens, wherein the plurality of connector tabs are each engaged with the plurality of lens fasteners,
wherein the eye protection lens is comprised of a flexible plastic and is molded to conform to a concave shape of an underside of the brim, wherein the eye protection lens is interchangeable, and wherein the brim fasteners and lens fasteners allow for interchanging eye protection lenses between a clear lens and a tinted lens.

2. The cap eye protection device of claim 1, wherein the cap is a baseball cap.

3. The cap eye protection device of claim 1, wherein the eye protection lens is tinted.

4. The cap eye protection device of claim 1, wherein the eye protection lens is selected from clear, mirrored tinted, yellow tint, red tint, orange tint, blue tint, light smoke, dark smoke and/or a custom design tint.

5. The cap eye protection device of claim 1, wherein the eye protection lens is treated for ultraviolet protection.

6. The cap eye protection device of claim 1, wherein the eye protection lens is shaped to have a profile accommodating a user's nose.

7. The cap eye protection device of claim 1, further comprising that eye protection lens is comprised of a flexible plastic and the top edge is molded to conform to a concave shape of an underside of the brim to reduce or eliminate debris, wind and/or sunlight from penetrating a seam between the eye protection lens and the underside of the brim.

8. The cap eye protection device of claim 1, further comprising that eye protection lens is comprised of a flexible plastic and the surface is molded to conform to about a curvature of a rim of a cap.

9. The cap eye protection device of claim 1, wherein the brim and eye protection lens connect via three pairs of fasteners and three connector tabs.

10. The cap eye protection device of claim 1, wherein the connector tabs each comprise a mounting hole capable of engaging with a fastener.

11. The cap eye protection device of claim 1, wherein the connector tabs are aligned at about a 90 degree angle relative to the eye protection lens.

12. The cap eye protection device of claim 1, wherein the brim fasteners and the corresponding lens fasteners are metal snaps, wherein the lens fasteners are capable of being engaged and disengaged with the brim fasteners.

13. A kit comprising a cap eye protection device comprising:
a) a cap member comprising a brim with a plurality of brim fasteners capable of engaging with a plurality of fasteners of an eye protection lens on an underside of the brim; and
b) a transparent eye protection lens comprising a plurality of lens fasteners capable of engaging with the plurality of brim fasteners, and a plurality of connector tabs formed on a top side of the eye protection lens, wherein the plurality of connector tabs are each engaged with the plurality of lens fasteners,
wherein the eye protection lens is comprised of a flexible plastic and is molded to conform to a concave shape of an underside of the brim, wherein the eye protection lens is interchangeable, and wherein the brim fasteners and lens fasteners allow for interchanging eye protection lenses between a clear lens and a tinted lens.

14. The kit of claim 13, wherein the eye protection lens is a plurality of lenses selected from clear, mirrored tinted, yellow tint, red tint, orange tint, blue tint, light smoke, dark smoke and/or a custom design tint.

15. The kit of claim 13, wherein the eye protection lens is treated for ultraviolet protection.

16. The kit of claim 13, wherein the brim and eye protection lens connect via three pairs of fasteners and three connector tabs.

17. The kit of claim 13, wherein the connector tabs each comprise a mounting hole capable of engaging with a fastener.

18. The kit of claim 13, wherein the connector tabs are aligned at about a 90 degree angle relative to the eye protection lens.

19. The kit of claim 13, wherein the brim fasteners and the corresponding lens fasteners are metal snaps, wherein the lens fasteners are capable of being engaged and disengaged with the brim fasteners.
